(19) United States
(12) Patent Application Publication
(10) Pub. No.: US 2022/0031598 A1
(43) Pub. Date: Feb. 3, 2022

(54) Oral Care Compositions Containing Abrasives and Cocamidopropyl Betaine

(71) Applicant: Colgate-Palmolive Company, New York, NY (US)

(72) Inventors: Svenia SCHMID, Basel (CH); Jeannine LOETSCHER, Ettingen (CH); Turan MATUR, Binningen (CH); Jessie FONTERS, Belmont-sur-Lausanne (CH)

(73) Assignee: Colgate-Palmolive Company, New York, NY (US)

(21) Appl. No.: 17/392,722

(22) Filed: Aug. 3, 2021

Related U.S. Application Data
(60) Provisional application No. 63/060,337, filed on Aug. 3, 2020.

(51) Int. Cl. A61K 8/70; A61K 8/42; A61K 8/25; A61K 8/27; A61Q 11/00

(52) U.S. Cl. CPC A61K 8/70; A61K 8/42; A61K 8/25; A61K 2800/33; A61Q 11/00; A61K 2800/28; A61K 8/27

(57) ABSTRACT

Disclosed are oral care compositions comprising an abrasive, cocamidopropyl betaine and an amine fluoride (e.g., olaflur (N′-octadecyltrimethylenediamine-N,N,N′-tris(2-ethanol)-dihydrofluoride)) or amine base plus fluoride, wherein the abrasive is present in an amount of from 16% to 30% by weight of the composition and wherein cocamidopropyl betaine is present in an amount of from 1.1% to 2% by weight of the composition, as well as methods of using these compositions.

### BACKGROUND

Cocamidopropyl betaine (CAPB) is an amphoteric compound that is often used as a surfactant in oral care products. Because of its amphoteric nature, it is compatible with anionic, cationic and nonionic surfactants to boost foaming and to improve dispersion of various oral care formulations. Due to these properties, cocamidopropyl betaine has been used to improve foaming and dispersion of the toothpastes.

Abrasives in oral care compositions debride and physically scrub the external surface of the teeth. This scrubbing action removes organic biofilm (i.e., the pellicle) on the tooth surface that is formed primarily of salivary proteins, bacteria, and bacterial byproducts. It can be stained and discolored by foods such as coffee, tea and berries as well as by tobacco smoke, cationic compounds, and chromogenic bacteria. Such physical removal of the stained pellicle is a simple and effective means of removing the undesirable surface staining and discoloration which occur daily. Further, such physical removal of the pellicle also removes plaque bacteria on the pellicle surface, thereby minimizing the potential for gingivitis, periodontitis, and caries formation. However, oral compositions with high abrasiveness can potentially damage the enamel or oral tissue.

As such, there remains a need in the art for oral care compositions that improve the cleaning properties of a given formula without changing the intrinsic abrasive system.

### BRIEF SUMMARY

In one aspect, the invention provides an oral care composition, e.g., toothpaste or gel, which comprises an abrasive, cocamidopropyl betaine and an amine fluoride (e.g., olaflur (N′-octadecyltrimethylenediamine-N,N,N′-tris(2-ethanol)-dihydrofluoride)) or amine base plus fluoride, wherein the abrasive is present in an amount of from 16% to 30% by weight of the composition and wherein cocamidopropyl betaine is present in an amount of from 1.1% to 2% by weight of the composition. In some embodiments, cocamidopropyl betaine is present in an amount of from 1.2% to 2%, from 1.3% to 2%, from 1.1% to 1.8%, from 1.1% to 1.7%, from 1.1% to 1.6%, from 1.1% to 1.5%, from 1.1% to 1.4%, from 1.1% to 1.3%, from 1.1% to 1.2%, from 1.2% to 1.8%, from 1.2% to 1.7%, from 1.2% to 1.6%, from 1.2% to 1.5%, from 1.2% to 1.4%, from 1.2% to 1.3%, or from 1.3% to 1.4% by weight of the composition. In some embodiments, the abrasive is present in an amount of from 16% to 25%, from 16% to 20%, from 20% to 30%, or from 20% to 25% by weight of the composition. In some embodiments, the abrasive is a silica abrasive. In some embodiments, the composition is free or substantially free of anionic surfactants.

In another aspect, the invention provides a method comprising applying an effective amount of any of oral care compositions as disclosed herein to the oral cavity, e.g., by brushing, to a subject in need thereof, to (i) reduce or inhibit formation of dental caries, (ii) reduce, repair or inhibit pre-carious lesions of the enamel, (iii) reduce or inhibit demineralization and promote remineralization of the teeth, (iv) reduce hypersensitivity of the teeth, (v) reduce or inhibit gingivitis, (vi) promote healing of sores or cuts in the oral cavity, (vii) reduce levels of acid producing bacteria, (viii) reduce or inhibit microbial biofilm formation in the oral cavity, (ix) reduce or inhibit plaque formation in the oral cavity, (x) promote systemic health, or (xi) clean teeth and oral cavity.

In another aspect, the invention provides the use of cocamidopropyl betaine in the manufacture of an oral care composition, e.g., toothpaste or gel, comprising an abrasive and an amine fluoride (e.g., olaflur (N′-octadecyltrimethylenediamine-N,N,N′-tris(2-ethanol)-dihydrofluoride)) or amine base plus fluoride, e.g., any of oral care compositions as disclosed herein, wherein the abrasive is present in an amount of from 16% to 30% by weight of the composition and wherein cocamidopropyl betaine is present in an amount of from 1.1% to 2% by weight of the composition. In some embodiments, the composition is free or substantially free of anionic surfactants.

In another aspect, the invention provides the use of cocamidopropyl betaine in an oral care composition, e.g., toothpaste or gel, comprising an abrasive and an amine fluoride (e.g., olaflur (N′-octadecyltrimethylenediamine-N,N,N′-tris(2-ethanol)-dihydrofluoride)) or amine base plus fluoride, e.g., any of oral care compositions as disclosed herein, for improving the cleaning properties of the composition, e.g., increasing the pellicle cleaning ratio (PCR) of the composition, wherein the abrasive is present in an amount of from 16% to 30% by weight of the composition and wherein cocamidopropyl betaine is present in an amount of from 1.1% to 2% by weight of the composition. In some embodiments, the composition is free or substantially free of anionic surfactants.

Further areas of applicability of the present disclosure will become apparent from the detailed description provided hereinafter. It should be understood that the detailed description and specific examples, while indicating some typical aspects of the disclosure, are intended for purposes of illustration only and are not intended to limit the scope of the disclosure.

### DETAILED DESCRIPTION

The following description of various typical aspect(s) is merely exemplary in nature and is in no way intended to limit the disclosure, its application, or uses.

As used throughout, ranges are used as shorthand for describing each and every value that is within the range. Any value within the range can be selected as the terminus of the range.

Unless otherwise specified, all percentages and amounts expressed herein and elsewhere in the specification should be understood to refer to percentages by weight. The amounts given are based on the active weight of the material.

In the present invention, it has been found that oral care formulations with a higher level of cocamidopropyl betaine show significantly higher pellicle cleaning ratio (PCR) values compared to formulations with the same backbone but a lower cocamidopropyl betaine content. Without intending to be bound to theory, it is believed that the increased PCR values caused by higher cocamidopropyl betaine level might result from better dispersion of abrasive particles on the surface of the teeth. Thus, the use of an increased amount of cocamidopropyl betaine may improve the cleaning properties of a given formula without changing the intrinsic abrasive system.

The present invention provides, in an aspect, an oral care composition (Compositions 1.0), e.g., toothpaste or gel, comprising an abrasive, cocamidopropyl betaine and an amine fluoride (e.g., olaflur (N′-octadecyltrimethylenediamine-N,N,N′-tris(2-ethanol)-dihydrofluoride)) or amine base plus fluoride, wherein the abrasive is present in an amount of from 16% to 30% by weight of the composition and wherein cocamidopropyl betaine is present in an amount of from 1.1% to 2% by weight of the composition.

For example, the invention includes:
1.1. Composition 1.0, wherein cocamidopropyl betaine is present in an amount of from 1.2% to 2%, from 1.3% to 2%, from 1.1% to 1.8%, from 1.1% to 1.7%, from 1.1% to 1.6%, from 1.1% to 1.5%, from 1.1% to 1.4%, from 1.1% to 1.3%, from 1.1% to 1.2%, from 1.2% to 1.8%, from 1.2% to 1.7%, from 1.2% to 1.6%, from 1.2% to 1.5%, from 1.2% to 1.4%, from 1.2% to 1.3%, or from 1.3% to 1.4% by weight of the composition.
1.2. Any of the preceding compositions, wherein cocamidopropyl betaine is present in an amount of from 1.3% to 1.4% by weight of the composition.
1.3. Any of the preceding compositions, wherein the abrasive is selected from silica abrasives, or abrasives such as sodium metaphosphate, potassium metaphosphate, aluminum silicate, calcined alumina, bentonite or other siliceous materials, and combinations thereof.
1.4. Any of the preceding compositions, wherein the abrasive comprises or is a silica abrasive.
1.5. Any of the preceding compositions, wherein the abrasive is present in an amount of from 16% to 25%, from 16% to 20%, from 20% to 30%, or from 20% to 25% by weight of the composition.
1.6. Any of the preceding compositions, wherein the abrasive is present in an amount of from 20% to 30% by weight of the composition.
1.7. Any of the preceding compositions having a pellicle cleaning ratio (PCR) of from 60 to 150, e.g., from 75 to 105, from 75 to 100, from 80 to 100, from 85 to 100, from 90 to 95, from 75-80, or from 95 to 100.
1.8. Any of the preceding compositions, wherein the amine fluoride or amine base plus fluoride is present in an amount sufficient to supply 25 ppm to 5.000 ppm of fluoride ions, generally at least 500 ppm. e.g., 500 to 2000 ppm, e.g., 1000 ppm to 1600 ppm, e.g., 1450 ppm.
1.9. Composition 1.8, wherein the amine fluoride is olaflur (N′-octadecyltrimethylenediamine-N,N,N′-tris(2-ethanol)-dihydrofluoride).
1.10. Any of the preceding compositions, wherein the composition is free or substantially free of sodium lauryl sulfate.
1.11. Any of the preceding compositions, wherein the composition is free or substantially free of alkyl sulfate salts.
1.12. Any of the preceding compositions, wherein the composition is free or substantially free of anionic surfactants.
1.13. Any of the preceding compositions, wherein the composition does not comprise any surfactant other than cocamidopropyl betaine.
1.14. Any of the preceding compositions, wherein the composition comprises a basic amino acid in free or salt form.
1.15. Composition 1.14, wherein the basic amino acid comprises one or more of arginine, lysine, citrulline, ornithine, creatine, histidine, diaminobutyric acid, diaminopropionic acid, salts thereof, or combinations thereof.
1.16. Composition 1.14 or 1.15, wherein the basic amino acid has the L-configuration.
1.17. Any of Compositions 1.14 to 1.16, wherein the basic amino acid is present in an amount of from 1% to 15%, e.g., from 1% to 10%, from 1% to 5%, from 1% to 3%, from 1% to 2%, from 1.2% to 1.8%, from 1.4% to 1.6%, or about 1.5% by weight of the composition, being calculated as free base form.
1.18. Any of Compositions 1.14 to 1.17, wherein the basic amino acid comprises arginine.
1.19. Any of Compositions 1.14 to 1.18, wherein the basic amino acid comprises L-arginine.
1.20. Any of Compositions 1.14 to 1.19, wherein the basic amino acid comprises arginine bicarbonate, arginine phosphate, arginine sulfate, arginine hydrochloride or combinations thereof, optionally wherein the basic amino acid is arginine bicarbonate.
1.21. Any of the preceding compositions, wherein the composition comprises a zinc ion source.
1.22. Composition 1.21, wherein the zinc ion source is selected from the group consisting of zinc oxide, zinc sulfate, zinc chloride, zinc citrate, zinc lactate, zinc gluconate, zinc malate, zinc tartrate, zinc carbonate, zinc phosphate and a combination thereof.
1.23. Any of Composition 1.21 to 1.22, wherein the zinc ion source is present in an amount of from 0.01% to 5%, e.g., 0.1% to 4%, or 0.5% to 3%, by weight of the composition.
1.24. Any of Composition 1.21 to 1.23, wherein the zinc ion source is selected from the group consisting of zinc oxide, zinc citrate, and a combination thereof, optionally wherein the zinc ion source is a combination of zinc oxide and zinc citrate.
1.25. Any of Composition 1.21 to 1.24, wherein zinc oxide is present in an amount of 0.5% to 2%, e.g., 0.5% to 1.5%, or about 1% by weight of the composition.
1.26. Any of Composition 1.21 to 1.25, wherein zinc citrate is present in an amount of 0.1% to 1%, 0.25 to 0.75%, or about 0.5% by weight of the composition.
1.27. Any of the preceding compositions, wherein the composition comprises a thickener.
1.28. Composition of 1.27, wherein the thickener comprises hydroxyethylcellulose.
1.29. Composition 1.27 or 1.28, wherein the thickener comprises a thickening silica, optionally wherein the thickening silica is present in an amount of from 2% to 10%, from 5% to 10%, from 6% to 8% or about 7%, by weight of the composition.
1.30. Any of the preceding compositions, wherein the composition comprises a humectant, optionally wherein the humectant is selected from sorbitol, glycerin and a mixture thereof.
1.31. Composition 1.30, wherein the humectant comprises glycerin, optionally wherein glycerin is present in an amount of from 15% to 40%, from 15% to 30%, from 20% to 40%, from 30% to 40%, or from 15% to 20% by weight of the composition.
1.32. Composition 1.30 or 1.31, wherein the humectant comprises sorbitol, optionally wherein sorbitol is present in an amount of from 15% to 40%, from 15% to 30%, from 20% to 40%, from 30% to 40%, or from 15% to 20% by weight of the composition.
1.33. Any of the preceding compositions, wherein the composition comprises one or more soluble phosphate salts, e.g., selected from tetrasodium pyrophosphate (TSPP), sodium tripolyphosphate (STPP) and a combination thereof.
1.34. Any of the preceding compositions, wherein the composition comprises water, optionally wherein water is present in an amount of from 10% to 80%, from 20% to 60%, from 20 to 50%, from 20% to 40%, from 10% to 30%, from 20% to 30%, from 30% to 50%, from 30% to 45%, from 30% to 40%, or from 25% to 35% by weight of the composition.
1.35. Any of the preceding compositions, wherein the abrasive is a silica abrasive, wherein the amine fluoride is olaflur (N′-octadecyltrimethylenediamine-N,N,N′-tris(2-ethanol)-dihydrofluoride), and wherein the composition is free or substantially free of anionic surfactants.
1.36. Any of the preceding compositions, wherein the composition is a toothpaste or gel.
1.37. Any of the preceding compositions, wherein the composition is a toothpaste.
1.38. Any of the preceding compositions for use in (i) reducing or inhibiting formation of dental caries, (ii) reducing, repairing or inhibiting pre-carious lesions of the enamel, (iii) reducing or inhibiting demineralization and promote remineralization of the teeth, (iv) reducing hypersensitivity of the teeth, (v) reducing or inhibiting gingivitis, (vi) promoting healing of sores or cuts in the oral cavity, (vii) reducing levels of acid producing bacteria, (viii) reducing or inhibiting microbial biofilm formation in the oral cavity, (ix) reducing or inhibiting plaque formation in the oral cavity, (x) promoting systemic health, or (xi) cleaning teeth and oral cavity.

The oral care composition of the invention can be in the form of any oral care formulations, including dentifrice, toothpaste, gel, powder, cream, strip, gum, bead, film, floss or any other known in the art. In some embodiments, the oral care composition is a toothpaste or gel. In some embodiments, the oral care composition is a toothpaste.

The oral care composition of the invention may be a single phase oral care composition. For example, all the components of the oral care composition may be maintained together with one another in a single phase and/or vessel. For example, all the components of the oral care composition may be maintained in a single phase, such as a single homogenous phase. In another embodiment, the oral care composition may be a multi-phase oral care composition.

The oral care composition of the invention may contain an orally acceptable carrier. As used herein, an “orally acceptable carrier” refers to a material or combination of materials that are safe for use in the compositions of the invention, commensurate with a reasonable benefit/risk ratio. Such materials include but are not limited to, for example, water, humectants, ionic active ingredients, buffering agents, anticalculus agents, abrasive polishing materials, peroxide sources, alkali metal bicarbonate salts, surfactants, titanium dioxide, coloring agents, flavor systems, sweetening agents, antimicrobial agents, herbal agents, desensitizing agents, stain reducing agents, and mixtures thereof. Such materials are well known in the art and are readily chosen by one skilled in the art based on the physical and aesthetic properties desired for the compositions being prepared. In some embodiment, the orally acceptable carrier may include an orally acceptable solvent. Illustrative solvents may include, but are not limited to, one or more of ethanol, phenoxyethanol, isopropanol, water, cyclohexane, methyl glycol acetate, benzyl alcohol, or the like, or any mixture or combination thereof. In a particular embodiment, the orally acceptable solvent includes benzyl alcohol.

Water may be present in the oral compositions of the invention. Water employed in the preparation of commercial oral compositions should be deionized and free of organic impurities. Water commonly makes up the balance of the compositions and includes about 10% to about 80%, about 20% to about 60%, about 20% to 50%, about 20% to 40%, about 10% to about 30%, about 20% to 30%, about 30% to about 50%, about 30% to about 45%, about 30% to about 40%, or about 25% to 35% by weight of the oral compositions. This amount of water includes the free water which is added plus that amount which is introduced with other materials such as with sorbitol or any components of the invention.

The oral care composition of the invention includes one or more abrasives or an abrasive system including one or more abrasives. The abrasives, e.g., silica abrasives, may be present in an amount of from 16% to 30%, e.g., from 16% to 25%, from 16% to 20%, from 20% to 30%, or from 20% to 25% by weight of the composition. As used herein, the term “abrasive” may also refer to materials commonly referred to as “polishing agents”. Any orally acceptable abrasive may be used, but preferably, type, fineness (particle size), and amount of the abrasive may be selected such that the tooth enamel is not excessively abraded in normal use of the oral care composition. The one or more abrasives may have a particle size or D50 of less than or equal to about 10 μm, less than or equal to about 8 μm, less than or equal to about 5 μm, or less than or equal to about 3 μm. The one or more abrasives may have a particle size or D50 of greater than or equal to about 0.01 μm, greater than or equal to about 0.05 μm, greater than or equal to about 0.1 μm, greater than or equal to about 0.5 μm, or greater than or equal to about 1 μm. Illustrative abrasives may include, but are not limited to, metaphosphate compounds, phosphate salts (e.g., insoluble phosphate salts), such as sodium metaphosphate, potassium metaphosphate, calcium pyrophosphate, magnesium orthophosphate, trimagnesium orthophosphate, tricalcium phosphate, dicalcium phosphate dihydrate, anhydrous dicalcium phosphate, calcium carbonate (e.g., precipitated calcium carbonate and/or natural calcium carbonate), magnesium carbonate, hydrated alumina, silica, zirconium silicate, aluminum silicate including calcined aluminum silicate, polymethyl methacrylate, or the like, or mixtures and combinations thereof.

In some embodiments, the oral care composition comprises a silica abrasive. In some embodiments, the oral care composition comprises a calcium-free silica abrasive.

In some embodiments, the oral care composition of the invention comprises a calcium-containing abrasive (e.g., calcium carbonate). In some embodiments, the calcium-containing abrasive is selected from calcium carbonate, calcium phosphate (e.g., dicalcium phosphate dihydrate), calcium sulfate, and combinations thereof. In some embodiments, the oral care composition comprises calcium carbonate as an abrasive. In one embodiment, the oral care composition comprises precipitated calcium carbonate or natural calcium carbonate. Precipitated calcium carbonate may be preferred over natural calcium carbonate.

The oral care composition of the invention comprises a surfactant system comprising cocamidopropyl betaine, wherein cocamidopropyl betaine is present in an amount of from 1.1% to 2% by weight of the composition. In some embodiments, cocamidopropyl betaine is present in an amount of from 1.1% to 2%, from 1.2% to 2%, from 1.3% to 2%, from 1.1% to 1.8%, from 1.1% to 1.7%, from 1.1% to 1.6%, from 1.1% to 1.5%, from 1.1% to 1.4%, from 1.1% to 1.3%, from 1.1% to 1.2%, from 1.2% to 1.8%, from 1.2% to 1.7%, from 1.2% to 1.6%, from 1.2% to 1.5%, from 1.2% to 1.4%, from 1.2% to 1.3%, or from 1.3% to 1.4% by weight of the composition.

Any cocamidopropyl betaine (CAPB) products may be used in the preparation of the oral care composition of the invention. Common commercial CAPB products are aqueous solutions in concentrations of about 30% CAPB. Higher concentrated CAPB solutions, e.g., Tego® Betain F 50, which contains about 38% CAPB, may be used. The amount of CAPB disclosed in this disclosure refers to the amount of CAPB present in the composition but not the amount of CAPB solution added into the composition. For example, the amount of CAPB present in a composition containing 3% Tego® Betain F 50 is regarded to be about 1.14% by weight of the composition in this disclosure.

In some embodiments, the oral care composition of the invention is free or substantially free of sodium lauryl sulfate. In some embodiments, the composition is free or substantially free of alkyl sulfate salts. In some embodiments, the composition is free or substantially free of anionic surfactants. As used herein, “substantially free” of a material may refer to a composition where the material is present in an amount of less than 0.1 weight %, less than 0.05 weight %, less than 0.01 weight %, less than 0.005 weight %, less than 0.001 weight %, or less than 0.0001 weight % based on a total weight of the composition. In some embodiments, the composition does not comprise any surfactant other than cocamidopropyl betaine.

In other embodiments, the oral care composition of the invention may comprise additional surfactants other than cocamidopropyl betaine. Additional surfactants, which may be anionic, cationic or nonionic, and are known for use in oral care compositions, may be included in the oral care composition of the invention. Suitable anionic surfactants include without limitation water-soluble salts of C₈₋₂₀ alkyl sulfates, sulfonated monoglycerides of C₈₋₂₀ fatty acids, sarcosinates, taurates and the like. Illustrative examples of these and other classes include sodium lauryl sulfate, sodium lauryl ether sulfate, ammonium lauryl sulfate, ammonium lauryl ether sulfate, sodium cocoyl monoglyceride sulfonate, sodium lauryl sarcosinate, sodium lauryl isoethionate, sodium laureth carboxylase, and sodium dodecyl benzenesulfonate. In some embodiments, the additional surfactant other than CAPB may be a non-ionic block copolymer. In some embodiments, the non-ionic block copolymer is a poly(propylene oxide)/poly(ethylene oxide) copolymer. In some embodiments, the copolymer has a polyoxypropylene molecular mass of from 3000 to 5000 g/mol and a polyoxyethylene content of from 60 to 80 mol %. In some embodiments, the non-ionic block copolymer is a poloxamer. In some embodiments, the non-ionic block copolymer is selected from: Poloxamer 338, Poloxamer 407, Poloxamer, 237, Poloxamer, 217, Poloxamer 124, Poloxamer 184, Poloxamer 185, and a combination of thereof.

In some embodiments, the oral care composition of the invention may have a pellicle cleaning ratio (PCR) of from 60 to 150, e.g., from 75 to 105, from 75 to 100, from 80 to 100, from 85 to 100, from 90 to 95, from 75-80, or from 95 to 100, PCR is a method known by those of skill in the art to measure the efficacy of removing tooth stains relative to a standard. For example, PCR of a composition may be measured by the method described in Example 1 of this disclosure.

The oral care composition of the invention includes an amine fluoride (e.g., olaflur (N′-octadecyltrimethylenediamine-N,N,N′-tris(2-ethanol)-dihydrofluoride)) or amine base plus fluoride. In some embodiment, the amine fluoride is olaflur (N′-octadecyltrimethylenediamine-N,N,N′-tris(2-ethanol)-dihydrofluoride). The amine fluoride or amine base plus fluoride may be present in an amount sufficient to supply 25 ppm to 5.000 ppm of fluoride ions, generally at least 500 ppm, e.g., 500 to 2000 ppm, e.g., 1000 ppm to 1600 ppm, e.g., 1450 ppm.

In some embodiments, the oral care composition of the invention comprises a basic amino acid in free or salt form. The basic amino acids which can be used in the compositions include not only naturally occurring basic amino acids, such as arginine, lysine, and histidine, but also any basic amino acids having a carboxyl group and an amino group in the molecule, which are water-soluble and provide an aqueous solution with a pH of about 7 or greater. Accordingly, basic amino acids include, but are not limited to, arginine, lysine, citrulline, ornithine, creatine, histidine, diaminobutyric acid, diaminopropionic acid, salts thereof or combinations thereof. In a particular embodiment, the basic amino acids are selected from arginine, lysine, citrulline, and ornithine. The basic amino acids of the oral care composition may generally be present in the L-form or L-configuration. The basic amino acids may be provided as a salt of a di- or tri-peptide including the amino acid. In some embodiments, at least a portion of the basic amino acid present in the oral care composition is in the salt form. In some embodiments, the basic amino acid is arginine, for example, L-arginine, or a salt thereof. Arginine may be provided as free arginine or a salt thereof. For example, Arginine may be provided as arginine phosphate, arginine hydrochloride, arginine sulfate, arginine bicarbonate, or the like, and mixtures or combinations thereof. The basic amino acid may be provided as a solution or a solid. For example, the basic amino acid may be provided as an aqueous solution. In some embodiment, the amino acid includes or is provided by an arginine bicarbonate solution. For example, the amino acid may be provided by an about 40% solution of the basic amino acid, such as arginine bicarbonate or alternatively called as arginine carbamate. In some embodiments, the basic amino acid is present in an amount of from 1% to 15%, e.g., from 1% to 10%, from 1% to 5%, from 1% to 3%, from 1% to 2%, from 1.2% to 1.8%, from 1.4% to 1.6%, or about 1.5% by weight of the composition, being calculated as free base form.

In some embodiments, the oral care composition of the invention comprises a zinc ion source. The zinc ion source may be or include a zinc ion and/or one or more zinc salts. For example, the zinc salts may at least partially dissociate in an aqueous solution to produce zinc ions. Illustrative zinc salts may include, but are not limited to, zinc lactate, zinc oxide, zinc chloride, zinc phosphate, zinc citrate, zinc acetate, zinc borate, zinc butyrate, zinc carbonate, zinc formate, zinc gluconate, zinc glycerate, zinc glycolate, zinc picolinate, zinc propionate, zinc salicylate, zinc silicate, zinc stearate, zinc tartrate, zinc undecylenate, and mixtures thereof. In some embodiments, the zinc ion source is present in an amount of from 0.01% to 5%, e.g., 0.1% to 4%, or 1% to 3%, by weight of the composition.

In some embodiments, the zinc ion source is selected from zinc oxide, zinc citrate, and a combination thereof. Zinc oxide may be present in an amount of 0.5% to 2%, e.g., 0.5% to 1.5%, or about 1% by weight of the composition. Zinc citrate may be present in an amount of 0.1% to 1%, 0.25% to 0.75%, about 0.5% by weight of the composition by weight of the composition. In some embodiments, the composition comprises zinc oxide and zinc citrate. The composition may comprise zinc oxide in an amount of 0.5% to 2%, e.g., 0.5% to 1.5%, about 1% or about 1.2% by weight of the composition and zinc citrate in an amount of 0.1% to 1%, 0.25% to 0.75%, about 0.5% by weight of the composition. In certain embodiments, the composition comprises zinc oxide in an amount of about 1% by weight of the composition and zinc citrate in an amount of about 0.5% by weight of the composition.

The oral care composition of the invention may include thickeners. Suitable thickeners may be any orally acceptable thickener or thickening agent configured to control the viscosity of the oral care composition. Illustrative thickeners may be or include, but are not limited to, colloidal silica, fumed silica, a cross-linked polyvinylpyrrolidone (PVP) polymer, cross-linked polyvinylpyrrolidone (PVP), or the like, or mixtures or combinations thereof. In some embodiments, the thickening system includes a cross-linked polyvinylpyrrolidone (PVP) polymer. The thickening system may also include POLYPLASDONE® XL 10F, which is commercially available from Ashland Inc. of Covington, Ky. Illustrative thickeners may also be or include, but are not limited to, carbomers (e.g., carboxyvinyl polymers), carrageenans (e.g., Irish moss, carrageenan, iota-carrageenan, etc.), high molecular weight polyethylene glycols (e.g., CARBOWAX®, which is commercially available from The Dow Chemical Company of Midland, Mich.), cellulosic polymers, hydroxyethylcellulose, carboxymethylcellulose, and salts thereof (e.g., CMC sodium), natural gums (e.g., karaya, xanthan, gum arabic, and tragacanth), colloidal magnesium aluminum silicate, thickening silicas or the like, or mixtures or combinations thereof. Thickeners particularly suitable of use in the oral care composition of the invention include natural and synthetic gums and colloids. In some embodiments, the thickener comprises hydroxyethylcelluose. In some embodiments, the thickener comprises at least one gum selected from carrageenan and xanthan gum. In some embodiments, the thickener comprises a thickening silica, optionally wherein the thickening silica is present in an amount of from 2% to 10%, from 5 to 10%, from 6% to 8% or about 7%, by weight of the composition.

In some embodiments, the oral care compositions of the invention may include one or more humectants. Humectants can reduce evaporation and also contribute towards preservation by lowering water activity and can also impart desirable sweetness or flavor to compositions. Illustrative humectants may be or include, but are not limited to, glycerin, propylene glycol, polyethylene glycol, sorbitol, xylitol, or the like, or any mixture or combination thereof. In a preferred embodiment, the orally acceptable vehicle may be or include, but is not limited to, glycerin or sorbitol. In some embodiments, the humectant is selected from glycerin, sorbitol and a combination thereof. In some embodiments, the humectant may be present in an amount of from 20% to 60%, for example from 15% to 40%, from 15% to 35%, from 20% to 40%, from 30% to 50%, from 30% to 40%, or from 40% to 45%, by weight of the composition. In some embodiments, the composition comprises glycerin, optionally wherein glycerin is present in an amount of from 15% to 40%, from 20% to 40%, from 30% to 40%, or about 35% by weight of the composition. In some embodiments, the composition comprises sorbitol, optionally wherein sorbitol is present in an amount of from 15% to 40%, from 20% to 40%, from 30% to 40%, or about 35% by weight of the composition.

The oral care composition of the present invention may include a preservative. Suitable preservatives include, but are not limited to, sodium benzoate, potassium sorbate, methylisothiazolinone, paraben preservatives, for example methyl p-hydroxybenzoate, propyl p-hydroxybenzoate, and mixtures thereof.

The oral care composition of the present invention may include a sweetener such as, for example, saccharin, for example sodium saccharin, acesulfam, neotame, cyclamate or sucralose; natural high-intensity sweeteners such as thaumatin, stevioside or glycyrrhizin; or such as sorbitol, xylitol, maltitol or mannitol. One or more of such sweeteners may be present in an amount of from 0.005% to 5% by weight, for example 0.01% to 1%, for example 0.01% to 0.5%, by weight of the composition.

The oral care composition of the present invention may include a flavoring agent. Suitable flavoring agents include, but are not limited to, essential oils and various flavoring aldehydes, esters, alcohols, and similar materials, as well as sweeteners such as sodium saccharin. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. The flavoring agent is typically incorporated in the oral composition at a concentration of 0.01 to 3% by weight.

The oral care composition of the invention may include one or more pH modifying agents. For example, the oral care composition may include one or more acidifying agents and/or one or more basifying agents configured to reduce and/or increase the pH thereof, respectively. Illustrative acidifying agents and/or one or more basifying agents may be or include, but are not limited to, an alkali metal hydroxide, such as sodium hydroxide and/or potassium hydroxide, citric acid, hydrochloric acid, or the like, or combinations thereof.

The oral care composition of the invention may also include one or more buffering agents configured to control or modulate the pH within a predetermined or desired range. Illustrative buffering agents may include, but are not limited to, sodium bicarbonate, sodium phosphate, sodium carbonate, sodium acid pyrophosphate, sodium citrate, and mixtures thereof. Sodium phosphate may include monosodium phosphate (NaH₂PO₄), disodium phosphate (Na₂HPO₄), trisodium phosphate (Na₃PO₄), and mixtures thereof. In a typical embodiment, the buffering agent may be anhydrous sodium phosphate dibasic or disodium phosphate and/or sodium phosphate monobasic. In another embodiment, the buffering agent includes anhydrous sodium phosphate dibasic or disodium phosphate, and phosphoric acid (e.g., syrupy phosphoric acid; 85%-Food Grade).

The oral care composition of the invention may include anticalculus agents. Illustrative anticalculus agents may include, but are not limited to, phosphates and polyphosphates (e.g., pyrophosphates), polyaminopropanesulfonic acid (AMPS), hexametaphosphate salts, zinc citrate trihydrate, polypeptides, polyolefin sulfonates, polyolefin phosphates, diphosphonates. In some embodiments, the anticalculus agent includes tetrasodium pyrophosphate (TSPP), sodium tripolyphosphate (STPP), or a combination thereof.

The oral care composition of the invention may include an antioxidant. Any orally acceptable antioxidant may be used, including, but not limited to, butylated hydroxyanisole (BHA), butylated hydroxytoluene (BHT), vitamin A, carotenoids, vitamin E, flavonoids, polyphenols, ascorbic acid, herbal antioxidants, chlorophyll, melatonin, or the like, or combinations and mixtures thereof.

The oral care composition of the invention may include one or more pigments such as whitening pigments. In some embodiments, the whitening pigments include particles ranging in size from about 0.1 μm to about 10 μm with a refractive index greater than about 1.2. Suitable whitening agents include, without limitation, titanium dioxide particles, zinc oxide particles, aluminum oxide particles, tin oxide particles, calcium oxide particles, magnesium oxide particles, barium oxide particles, silica particles, zirconium silicate particles, mica particles, talc particles, tetracalcium phosphate particles, amorphous calcium phosphate particles, alpha-tricalcium phosphate particles, beta-tricalcium phosphate particles, hydroxyapatite particles, calcium carbonate particles, zinc phosphate particles, silicon dioxide particles, zirconium silicate particles, or the like, or mixtures and combinations thereof. The whitening pigment, such as titanium dioxide particles, may be present in an amount that is sufficient to whiten the toothpaste.

All ingredients for use in the compositions described herein should be orally acceptable. As used herein, “orally acceptable” may refer any ingredient that is present in a composition as described in an amount and form which does not render the composition unsafe for use in the oral cavity.

In another aspect, the present invention provides a method to (i) reduce or inhibit formation of dental caries, (ii) reduce, repair or inhibit pre-carious lesions of the enamel, (iii) reduce or inhibit demineralization and promote remineralization of the teeth, (iv) reduce hypersensitivity of the teeth, (v) reduce or inhibit gingivitis, (vi) promote healing of sores or cuts in the oral cavity, (vii) reduce levels of acid producing bacteria, (viii) reduce or inhibit microbial biofilm formation in the oral cavity, (ix) reduce or inhibit plaque formation in the oral cavity, (x) promote systemic health, or (xi) clean teeth and oral cavity, comprising applying an effective amount of any of oral care compositions as disclosed herein, e.g., any of Compositions 1 et seq., to the oral cavity of a subject in need thereof. The method may include contacting the oral care composition with water. The method may also include contacting the surface of the teeth with the oral care composition. Contacting the surface of the teeth with the oral care composition may include disposing the oral care composition (e.g., toothpaste) on a toothbrush and brushing the teeth with the toothbrush. The oral care composition may be applied and/or contacted with the surfaces of the teeth at predetermined intervals. For example, a daily basis, at least once a day, twice a day, or more, for multiple days, or alternatively every other day. In another example, the oral care composition may be applied and/or contacted with the surfaces of the teeth at least once a day, at least once every two days, at least once every three days, at least once every five days, at least once a week, at least once every two weeks, or at least once a month. The oral care composition thereof may be utilized for up to 2 weeks, up to 3 weeks, up to 4 weeks, up to 6 weeks, up to 8 weeks, or greater.

In another aspect, the invention provides the use of any of oral care compositions as disclosed herein, e.g., any of Compositions 1 et seq., to (i) reduce or inhibit formation of dental caries, (ii) reduce, repair or inhibit pre-carious lesions of the enamel, (iii) reduce or inhibit demineralization and promote remineralization of the teeth, (iv) reduce hypersensitivity of the teeth, (v) reduce or inhibit gingivitis, (vi) promote healing of sores or cuts in the oral cavity, (vii) reduce levels of acid producing bacteria, (viii) reduce or inhibit microbial biofilm formation in the oral cavity, (ix) reduce or inhibit plaque formation in the oral cavity, (x) promote systemic health, or (xi) clean teeth and oral cavity, in a subject in need thereof.

In another aspect, the invention provides the use of cocamidopropyl betaine in the manufacture of an oral care composition, e.g., toothpaste or gel, comprising an abrasive and an amine fluoride (e.g., olaflur (N′-octadecyltrimethylenediamine-N,N,N′-tris(2-ethanol)-dihydrofluoride)) or amine base plus fluoride, e.g., any of oral care compositions as disclosed herein, e.g., any of Compositions 1 et seq., wherein the abrasive is present in an amount of from 16% to 30% by weight of the composition and wherein cocamidopropyl betaine is present in an amount of from 1.1% to 2% by weight of the composition. In some embodiments, the composition is free or substantially free of anionic surfactants. In some embodiments, the abrasive comprises or is a silica abrasive.

The invention further provides the use of cocamidopropyl betaine in an oral care composition, e.g., toothpaste or gel, comprising an abrasive and an amine fluoride (e.g., olaflur (N′-octadecyltrimethylenediamine-N,N,N′-tris(2-ethanol)-dihydrofluoride)) or amine base plus fluoride, e.g., any of oral care compositions as disclosed herein, e.g., any of Compositions 1 et seq., for improving the cleaning properties of the composition, e.g., increasing the pellicle cleaning ratio (PCR) of the composition, wherein the abrasive is present in an amount of from 16% to 30% by weight of the composition and wherein cocamidopropyl betaine is present in an amount of from 1.1% to 2% by weight of the composition. In some embodiments, the composition is free or substantially free of anionic surfactants. In some embodiments, the abrasive comprises or is a silica abrasive.

### EXAMPLES

### Example 1

Four silica-based toothpastes having the formulations as indicated in Table 1 were prepared.

TABLE 1

| | Comp. I | Comp. II | Comp. III | Comp. IV |
|---|---|---|---|---|
| ingredient | (wt. %) | (wt. %) | (wt. %) | (wt. %) |
| Water | 42.54 | 40.94 | 33.97 | 31.97 |
| Sorbitol (70% solution) | 25 | 25 | 25 | 25 |
| Amine fluoride | 10 | 10 | 10 | 10 |
| (F = 1.4%) | | | | |
| Silica | 16 | 16 | 25 | 25 |
| Tego Betain F 50 | 1.5 | 3.0 | 1.5 | 3.5 |
| Flavor, sweetener, | Balance | Balance | Balance | Balance |
| preservative and colors | | | | |

In order to examine the effect of the amount of cocamidopropyl betaine on the cleaning efficacy of abrasive systems, two abrasive systems were tested. Compositions I and II contain 16% of a combination of 3 silicas in the same concentration. Compositions III and IV contain 25% of a combination of 2 silicas in the same concentration. Composition II and IV are identical to Composition I and Composition III, respectively, except that Compositions II and IV contains higher amounts of Tego® Betain F 50 than Compositions I and III: Compositions I and III contain 1.5% Tego® Betain F 50, while Compositions II and IV contain 3.0% and 3.5% Tego® Betain F 50, respectively. Tego® Betain F 50 is a concentrated cocamidopropyl betaine solution with high active concentration (about 38%). Thus, the amount of cocamidopropyl betaine in Compositions II and IV is about 1.14% and 1.33%, respectively.

To compare the cleaning efficacy of the compositions, pellicle cleaning ratio (PCR) was measured according to the protocol from Oral Health Research Institute, Indiana University. Briefly, the enamel specimens (bovine permanent central incisors) were embedded in a resin. They were exposed to a staining solution containing tea, coffee, FeCl₃, etc. After staining, specimens were rinsed, allowed to air dry, and refrigerated until used. The specimens were mounted on a mechanical brushing machine. The dentifrices were tested as slurries prepared by mixing 25 grams of dentifrice with 40 ml of deionized water. The specimens were brushed for 800 strokes. The mean decrement between the pre- and post-brushing stain scores was determined for the ADA Reference Material group and assigned a pellicle cleaning ratio (PCR) value of 100. A constant value was calculated by dividing the mean decrement of the ADA Reference Material into 100. The individual PCR value for each specimen was calculated by multiplying its individual decrement by the calculated constant. The larger the PCR value, the greater the amount of stained pellicle removed from the enamel surface in this test. The results are shown in Tables 2 and 3.

TABLE 2
PCR values of Compositions I and II

PCR

| Sample | Comp. I | Comp. II |
|---|---|---|
| 1 | 75.6 | 83.4 |
| 2 | 52 | 75.8 |
| 3 | 83.8 | 100.1 |
| 4 | 85.5 | 75.8 |
| 5 | 34.8 | 71 |
| 6 | 47.8 | 70.6 |
| 7 | 34.5 | 83.3 |
| 8 | 67.9 | 85.3 |
| 9 | 54.3 | 81 |
| 10 | 82.5 | 76.8 |
| 11 | 77 | 89.1 |
| 12 | 55.8 | 91.9 |
| 13 | 75.6 | 54.5 |
| 14 | 60.2 | 70.6 |
| 15 | 56.4 | 58.2 |
| 16 | 61.9 | 66.6 |
| Mean | 62.85 | 77.13 |

| | |
|---|---|
| P-value | 0.0084 |

TABLE 3
PCR values of Compositions III and IV

PCR

| Sample | Comp. III | Comp. IV |
|---|---|---|
| 1 | 93.2 | 108.5 |
| 2 | 98.1 | 98 |
| 3 | 91.8 | 110.8 |
| 4 | 94.1 | 99.5 |
| 5 | — | 100.2 |
| 6 | 63.2 | 115.2 |
| 7 | 84.2 | 90 |
| 8 | 91.5 | 96 |
| 9 | 96.6 | — |
| 10 | 72.2 | 88.7 |
| 11 | 82.6 | 90.1 |
| 12 | 91.5 | 98.4 |
| 13 | 103.3 | 94.2 |
| 14 | 89 | 91 |
| 15 | 87.4 | 96.4 |
| 16 | 70.8 | 94.4 |
| Mean | 87.3 | 98.09 |

| | |
|---|---|
| P-value | 0.0046 |

As shown in Tables 2 and 3, toothpaste formulations with a higher level of CAPB showed significantly higher PCR values compared to formulations with the same backbone but lower cocamidopropyl betaine content. Composition I and Composition II have the same abrasive system. An increased amount of Tego® Betain F 50 from 1.5% to 3.0% resulted in an elevation of the PCR value from 62.85 to 77.13 (Table 2). Similar effect was observed in toothpaste formulations having a different abrasive system (Composition III and IV). An increased amount of Tego® Betain F 50 from 1.5% to 3.5% resulted in an elevation of the PCR value from 87.30 to 98.08 (Table 3). These results demonstrate that the polishing properties of toothpastes can be improved by using a higher amount of cocamidopropyl betaine without changing the intrinsic abrasive system.

The present disclosure has been described with reference to exemplary embodiments. Although a limited number of embodiments have been shown and described, it will be appreciated by those skilled in the art that changes may be made in these embodiments without departing from the principles and spirit of the preceding detailed description. It is intended that the present disclosure be construed as including all such modifications and alterations insofar as they come within the scope of the appended claims or the equivalents thereof.

1. An oral care composition comprising an abrasive, cocamidopropyl betaine and an amine fluoride or amine base plus fluoride, wherein the abrasive is present in an amount of from 16% to 30% by weight of the composition and wherein cocamidopropyl betaine is present in an amount of from 1.1% to 2% by weight of the composition.

2. The composition of claim 1, wherein the composition is free or substantially free of anionic surfactants.

3. The composition of claim 1, wherein cocamidopropyl betaine is present in an amount of from 1.3% to 1.4% by weight of the composition.

4. The composition of claim 1, wherein the abrasive comprises a silica abrasive.

5. The composition of claim 1, wherein the abrasive is present in an amount of from 20% to 30% by weight of the composition.

6. The composition of claim 1, wherein the composition has a pellicle cleaning ratio (PCR) of from 60 to 150.

7. The composition of claim 1, wherein the amine fluoride is olaflur (N′-octadecyltrimethylenediamine-N,N,N′-tris(2-ethanol)-dihydrofluoride).

8. The composition of claim 1, wherein the composition comprises a basic amino acid in free or salt form.

9. The composition of claim 1, wherein the composition comprises a zinc ion source.

10. The composition of claim 1, wherein the oral care composition is a toothpaste or gel.

11. (canceled)

12. (canceled)
